(19) United States
(12) Patent Application Publication
(10) Pub. No.: US 2019/0201662 A1
(43) Pub. Date: Jul. 4, 2019

(54) OCCLUSION-RESISTANT CATHETER WITH OCCLUSION-RESISTANT TIP

(71) Applicant: Duke University, Durham, NC (US)

(72) Inventors: Nandan Lad, Durham, NC (US); Brian Bowman, Solana Beach, CA (US); Benjamin Arnold, Solana Beach, CA (US); Kristen Pena, Solana Beach, CA (US); Forrest Samuel, Solana Beach, CA (US)

(21) Appl. No.: 16/323,667

(22) PCT Filed: Aug. 10, 2017
(86) PCT No.: PCT/US2017/046197

§ 371 Date: Feb. 6, 2019

Related U.S. Application Data
(60) Provisional application No. 62/372,988, filed on Aug. 10, 2016.

(51) Int. Cl. A61M 25/00; A61L 29/08; A61L 29/16

(52) U.S. Cl. CPC A61M 25/007; A61L 29/085; A61M 27/002; A61L 2400/10; A61L 29/16

(57) ABSTRACT

An occlusion-resistant catheter, occlusion-resistant catheter tip, and methods of use are disclosed. In one embodiment, the occlusion-resistant catheter includes a hollow cannula defining a fluid passageway therethrough, the hollow cannula having a body portion with a first end and a second end, and an occlusion-resistant tip positioned at the first end of the body portion. The occlusion-resistant tip has at least one port that provides access to an interior lumen, and the interior lumen is in fluid communication with the fluid passageway of the body portion.

### CROSS REFERENCE TO RELATED APPLICATION

This application claims priority to U.S. Provisional Patent Application No. 62/372,988, filed Aug. 10, 2016, which are hereby incorporated by reference in its entirety.

### BACKGROUND

Ventriculoperitoneal (VP) shunt surgery is the predominant mode of therapy for patients with hydrocephalus, which is a build-up of fluid in the cavities deep within the brain. However, there are potential complications that may require multiple surgical procedures during a patient's lifetime. The most common reason for failure is proximal catheter obstruction by ingrowth of tissue. The catheter can become occluded by cellular debris in the cerebrospinal fluid, biofilm formation, or tissue proliferation in the catheter.

The ventricular tip of current shunt systems is often made of a sealed piece of silicone tubing with a series of holes in the sides. The fluid flows through these holes into the lumen of the tubing. When choroid plexus, or other brain tissue, grows into the holes, it tends to bridge in the lumen of the tubing causing an obstruction. When the lumen of the tube is completely obstructed, surgery is needed to replace the ventricular catheter.

Thus, there is a need for a low cost, safe, and consistent system that provides improved resistance to proximal catheter occlusion, thereby reducing the rate of surgical VP shunt revisions.

### SUMMARY

An occlusion-resistant catheter is disclosed. In one embodiment, the occlusion-resistant catheter includes a hollow cannula defining a fluid passageway therethrough, the hollow cannula including a body portion having a first end and a second end, and an occlusion-resistant tip positioned at the first end of the body portion, the occlusion-resistant tip having at least one port that provides access to an interior lumen, the interior lumen being in fluid communication with the fluid passageway of the body portion.

In another embodiment, a method of decreasing pressure in the brain of a subject comprising inserting into the brain of the subject an occlusion-resistant catheter as described herein such that the pressure is decreased in the brain of the subject.

In another aspect, a method of draining cerebrospinal fluid from the brain of a subject comprising inserting into the brain of the subject an occlusion-resistant catheter as described herein such that the cerebrospinal fluid is drained from the brain of the subject.

### BRIEF DESCRIPTION OF THE DRAWINGS

FIG. 1 is a perspective view of an occlusion-resistant catheter in accordance with one embodiment of the present disclosure.

FIG. 2 is a cross-section of the interior portion of the catheter shown in FIG. 1.

FIG. 3 is a side view of an occlusion-resistant tip of the catheter shown in FIG. 1.

FIG. 4A is a perspective view of an alternative embodiment of an occlusion-resistant tip of the present disclosure.

FIG. 4B is a side view of the occlusion-resistant tip shown in FIG. 4A.

FIG. 5A is a perspective view of an alternative embodiment of an occlusion-resistant tip of the present disclosure.

FIG. 5B is a side view of the occlusion-resistant tip shown in FIG. 5A.

FIG. 6A is a side view of another embodiment of an occlusion-resistant tip of the present disclosure.

FIG. 6B is an exploded view of the occlusion-resistant tip shown in FIG. 6A.

FIG. 7 is a perspective view with a cross-section of yet another embodiment of an occlusion-resistant tip of the present disclosure.

### DETAILED DESCRIPTION

For the purposes of promoting an understanding of the principles of the disclosure, reference will now be made to preferred embodiments and specific language will be used to describe the same. It will nevertheless he understood that no limitation of the scope of the disclosure is thereby intended, such alteration and further modifications of the disclosure as illustrated herein, being contemplated as would normally occur to one skilled in the art to which the disclosure relates.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject is a human patient that is in need of having fluid drained from an organ or tissue. In certain embodiments, the subject is a human patient suffering from hydrocephalus.

A catheter having unique tip geometry is disclosed. The tip provides improved resistance to occlusion and slows catheter obstruction. Such catheters will fit into the existing clinical pathway and procedure, require no extensive training, and will reduce shunt revisions. By preventing ingrowth of tissue, shunt malfunctions can be greatly reduced and obviate the need for repeated surgical procedures.

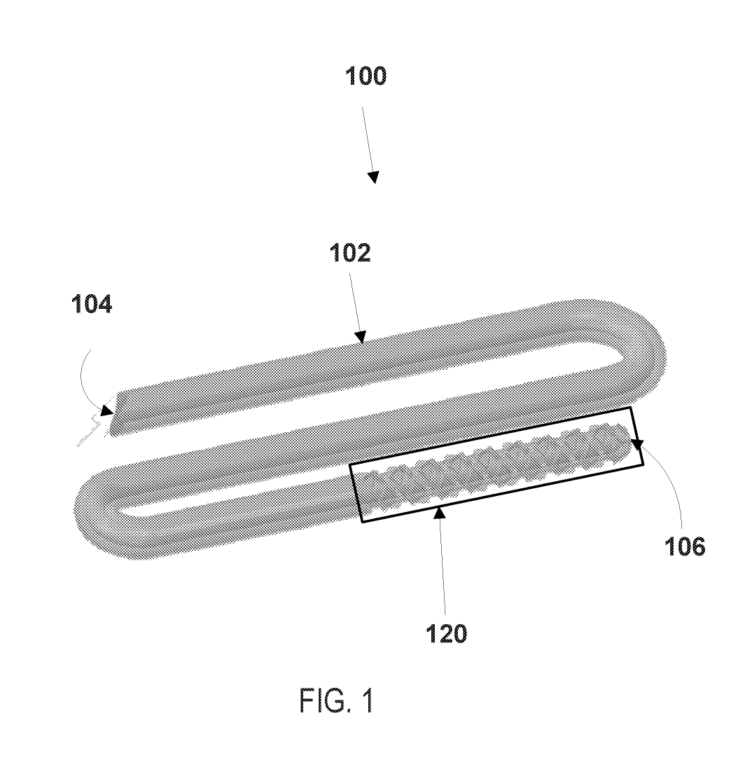

FIG. 1 shows an occlusion-resistant catheter 100 in accordance with one embodiment. As shown in FIG. 1, the occlusion-resistant catheter 100 comprises a hollow cannula 102 that has a first end 106 and a second end 104. The length of the hollow cannula 102 may be any length that is sufficient to provide for the insertion of the first end 106 of the cannula into the cranial cavity of a subject or patient, with the second end 104 connecting to a commercially available shunt or drainage system (not shown) to allow for drainage of cerebrospinal fluid from the brain of the subject. Generally, the hollow cannula 102 has a length of about 5 cm to about 40 cm. In some embodiments, the cannula 102 has a length of about 10 cm to about 35 cm.

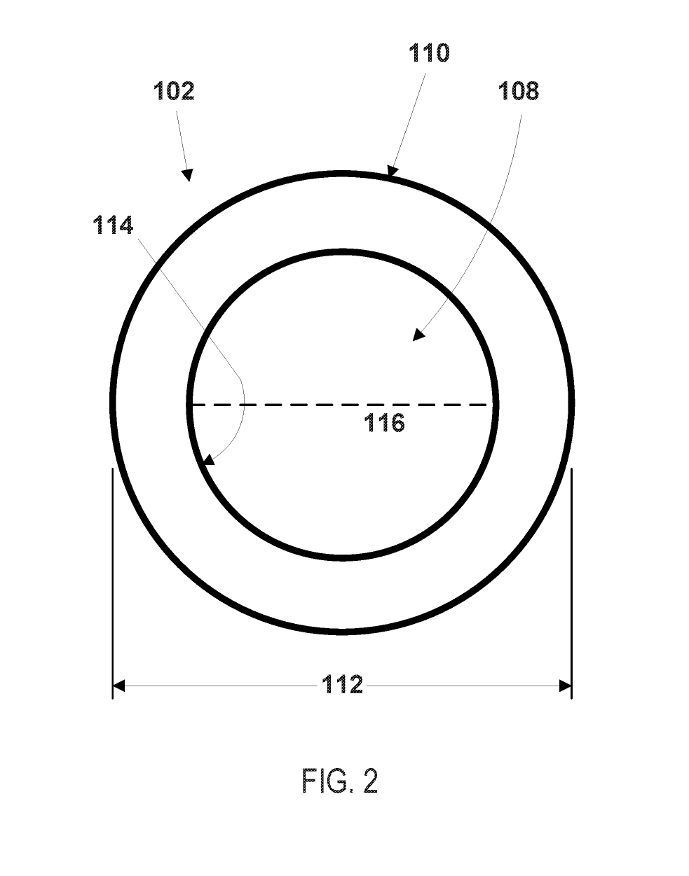

Referring to FIG. 2, the hollow cannula 102 defines a fluid passageway or lumen 108. The hollow cannula 102 comprises an exterior surface 110 that defines an exterior diameter 112 and an interior surface 114 that defines an interior diameter 116. The size of the cannula (outer and inner diameters) is of typical size for catheters used for shunt systems. Generally, the hollow cannula 102 comprises an outer diameter 112 of about 1.5 mm to about 4 mm. In certain embodiments, the outer diameter 112 is about 2 mm to about 3.5 mm. Generally, the hollow cannula 102 comprises an inner diameter 116 of about 0.5 mm to about 2 mm. In certain embodiments, the inner diameter 116 is about 1 mm to about 1.5 mm. The hollow cannula 102 may comprise any shape. In certain embodiments, the hollow cannula 102 is tubular or circular in shape.

Referring again to FIG. 1, the occlusion-resistant catheter 100 comprises an occlusion-resistant tip 120 (encircled) positioned at the second end 106 of the hollow cannula 102. The tip 120 is responsible for cerebrospinal fluid inflow, and comprises at least one entrance port leading to a central lumen that is in fluid communication with the interior diameter of the hollow catheter 102.

The occlusion-resistant tip 120 may comprise any length that is sufficient for allowing the catheter 100 to be inserted into the brain of a subject, and allow for drainage of the cerebrospinal fluid. Suitable lengths include, but are not limited to, about 0.5 cm to about 3.5 cm. In certain embodiments, the length of the occlusion-resistant tip 120 is about 1 cm to about 3 cm.

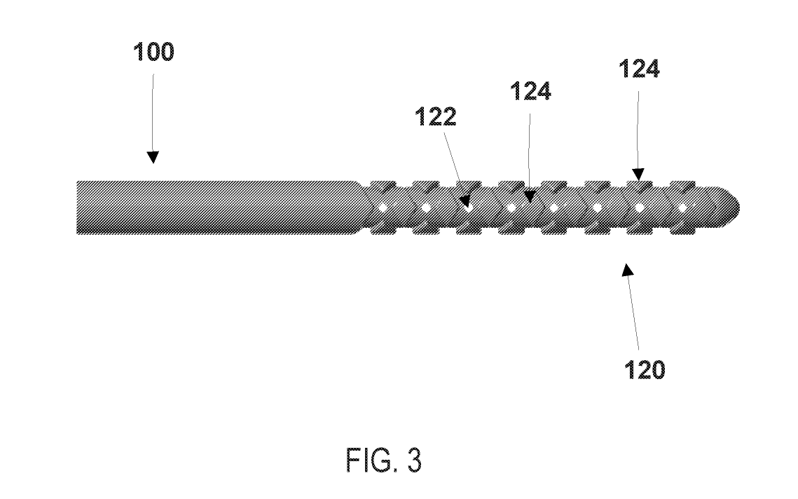

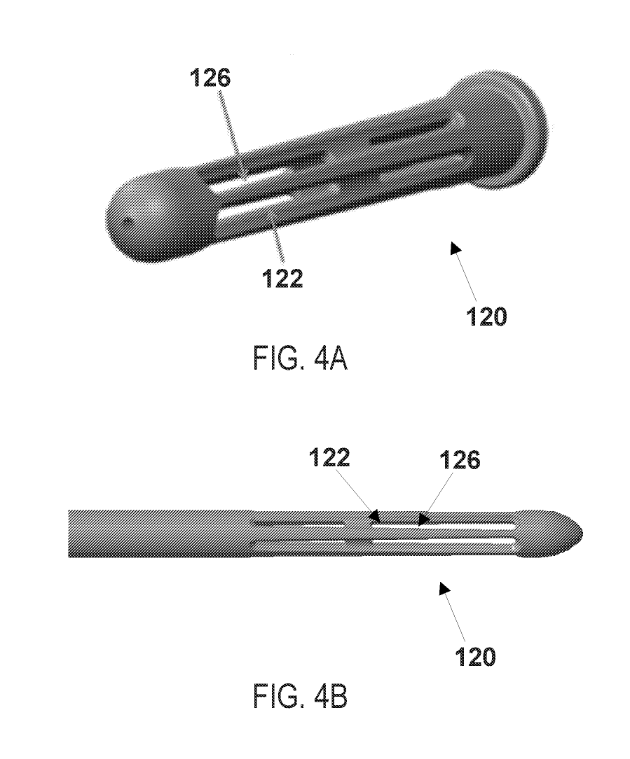

In some examples, and as shown in FIG. 3, the tip 120 includes at least one port 122 that provides access to an interior lumen 126 (shown in FIG. 4A) that is in fluid communication with the fluid passageway and interior diameter 116 of the hollow cannula 102. In one embodiment, the occlusion-resistant tip 120 may comprise a plurality of protrusions 124 positioned around a plurality of ports 122. The protrusions 124 on the tip 120 create a more tortuous path for the ingrowth of tissue, such as the choroid plexus, thereby reducing the occurrence of catheter occlusion. In sonic embodiments, the protrusions 124 may comprise a helical shape around the tip 120. In other embodiments, the protrusions 124 may comprise a spiral shape around the tip 120. In other embodiments, other shapes are possible as well.

The protrusions 124 may be of uniform or different sizes across the tip 120. Similarly, the protrusions 124 may be of uniform or different heights. In some embodiments, the protrusions may be 0.1 mm to about 20 mm in height. In other embodiments, the protrusions may be 0.5 mm to about 10 mm in height. Other heights are possible as well.

FIGS. 4A-B show another embodiment of an occlusion-resistant tip 120 of the catheter 100. In this embodiment, the plurality of ports 122 are in the shape of elongated slots. The elongated slots provide access for fluid to pass into the interior lumen 126 of the tip 120. The elongated slots may he of uniform lengths and/or sizes, or of varying lengths and/or sizes, In one embodiment, the elongated slots may be rectangular in shape. Alternatively, the ports 122 may comprise any elongated shape that allows for fluid to pass through. In some embodiments, the elongated ports may be recessed.

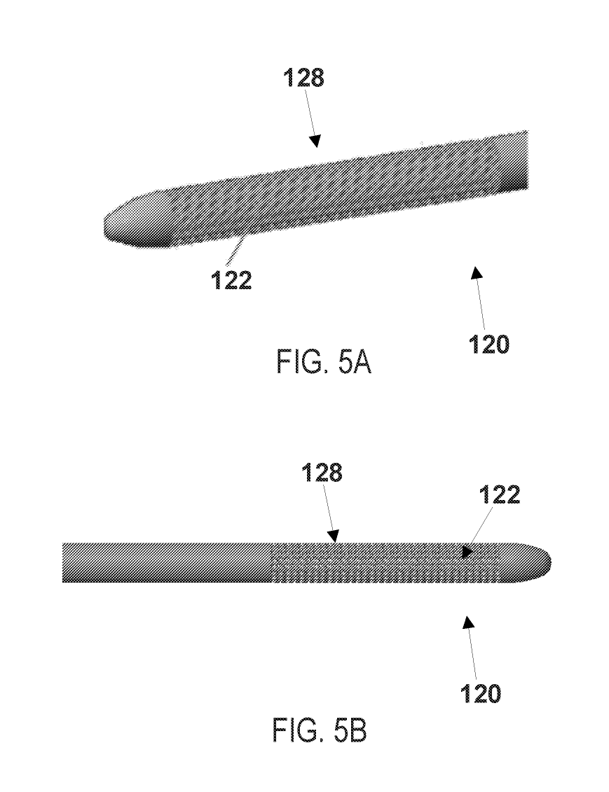

In another embodiment, and as shown in FIGS. 5A-B, the tip 120 includes a lattice structure 128, with a plurality of ports 122 being provided within the lattice. The ports 122 allow fluid to pass into the interior lumen 126 of the tip.

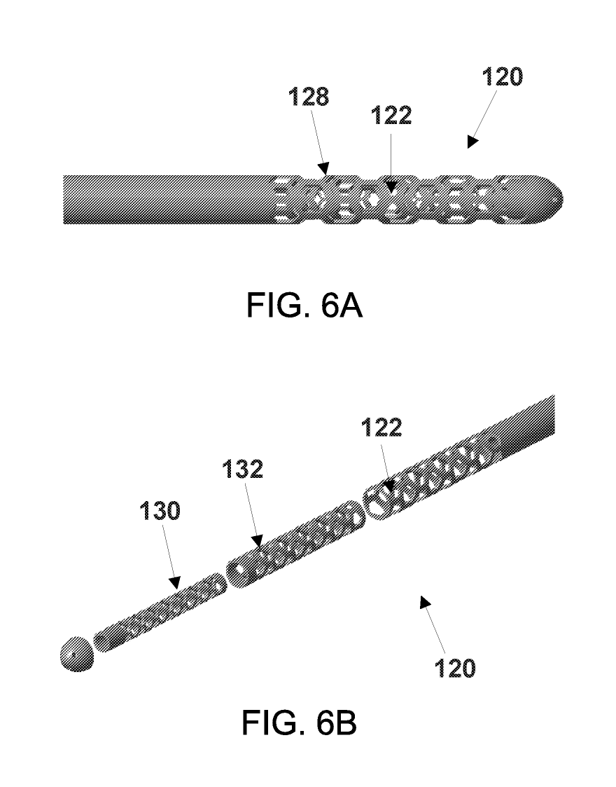

Referring to FIGS. 6A-B, another embodiment of the occlusion-resistant tip 120 is shown. The tip 120 includes a lattice structure 128 having a plurality of layers 130, 132. In one embodiment, each layer includes a plurality of ports 122, and the ports 122 in each layer are staggered, that is, they are not aligned with the layer above and below. Thus, a tortuous path is created for the fluid to flow into the catheter lumen.

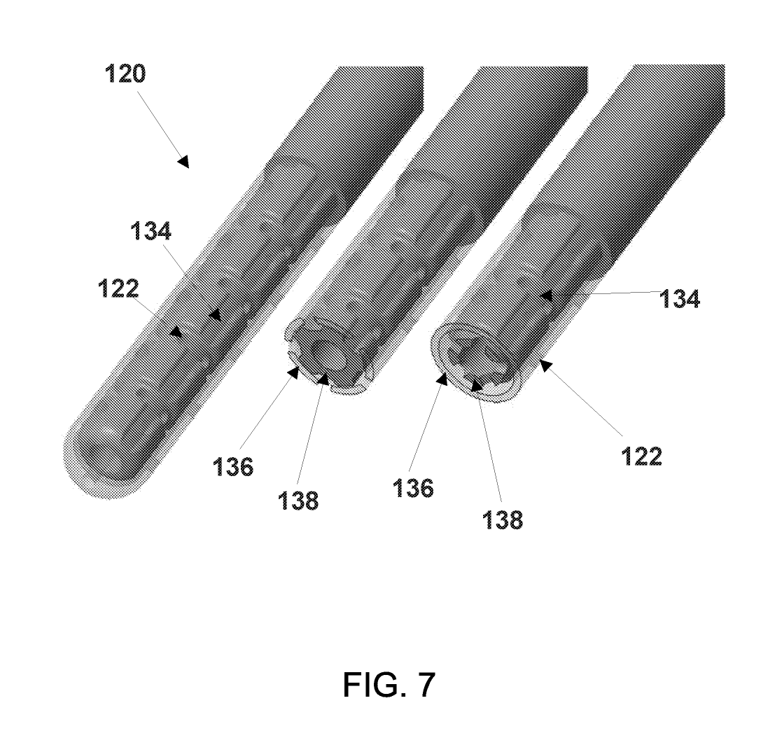

In yet another embodiment, as shown in FIG. 7, the occlusion-resistant tip 120 includes a plurality of recesses 134. The recesses 134 function to help channel fluid into a plurality of ports 122, which are positioned at the bottom of the recesses 134. The recesses 134 also allow for continual drainage of fluid should the catheter tip 120 be placed against a solid surface, such as tissue or bone, etc.

Additionally, the occlusion-resistant tip 120 shown in FIG. 8 may include one or more layers 136, 138 that are nested together. In such an embodiment, each layer defines an interior lumen, and comprises a plurality of recesses 134, each having a plurality of ports 122 positioned at the bottom thereof. The ports 122 then provide access for fluid to pass to the layer below, and eventually into the interior lumen of the tip 120.

The recesses 134 may comprise any number of shapes or sizes, including but not limited to, holes, parallel channels, intersecting channels, helical channels, channels having varying widths and lengths, and combinations thereof. In other embodiments, the recesses are physically shielded to protect the plurality of ports from being exposed to tissue. The shielding may comprise an overhang or other barrier that covers the port and thereby prevents tissue from growing into/around the port.

The occlusion-resistant catheter 100 and tip 120 may be made of materials having the following properties: high tensile strength; resistance to collapsing; flexibility; stability; ability to accept coating; ability to accept antibiotic or antimicrobial impregnation; ability to accept radiopaque additives; and biocompatible. High tensile strength allows the catheter to withstand the twisting and applied torque while being maneuvered through the brain tissue to the blockage, damaged area, or cavity when pushed through the blood vessel/tissue systems. Resistance to compression allows the catheter to maintain its shape, which maintains a flow path through the lumen of the catheter. In one embodiment, the material used has a high modulus and good kink resistance.

The occlusion-resistant catheter and tip may have flexibility for moving through the tissue systems. The stiffness may be the same or may vary along the length of the cannula or shaft. The amount of flexibility may be dependent on the type and function of the catheter, and can be readily determined by one skilled in the art.

The occlusion-resistant catheter may be comprised of a material with a low coefficient of friction. In some embodiments, the occlusion-resistant catheter and tip may use lubricious coatings on the inner or outer surface.

In some embodiments, the occlusion-resistant catheter may be comprised of a material that can accept antibiotic or antimicrobial coatings or impregnation. Additives may reduce the risk of infection.

The material selected for the occlusion-resistant catheter and tip preferably does not contain leachable additives that could cause failure in biocompatibility testing. Leachable additives could be cytotoxic or have systemic toxicity characteristics. Most commercially available materials typically have stabilizers or process aides. In addition, the material may be acceptable as a material for catheter tubing if it meets USP Class VI classifications. Materials meeting FDA's 21 CFR requirements for plastics in contact with food applications are another source for selection.

The material should also remain stable during storage and while in the body. The loss of properties during storage could cause failure when used. In storage and sterilization, materials could be exposed to moisture (humidity), heat, or light. Catheters with antibiotic or antimicrobial agents are often sterilized using moist heat. Some materials will degrade when exposed to these conditions.

Additionally, the material should be able to withstand chemical sterilization since catheters must he sterilized before they can be used in a subject. Examples of some chemical sterilization methods include, but are not limited to, ethylene oxide (EtO), Sterrad, Steris System 1, and Cidex OPA processes. EtO is another method often chosen for the sterilization of catheters. Additionally, since catheters are often exposed to a wide range of chemicals, in some embodiments, the occlusion-resistant catheter comprises a material, or combination of materials, that are able to withstand such exposure.

In another embodiment, the occlusion-resistant catheter 100 and occlusion-resistant tip 120 may be made of any biocompatible material suitable for medical use. In certain embodiments, the material, or combination of materials, meet the requirements as outlined above. Examples include, but are not limited to, (a) polyurethanes, including but not limited to, polycarbonate-based polyurethanes, polyether-based polyurethanes (e.g., aliphatic, aromatic, etc.), and thermoplastic polyurethanes (e.g., polyvinyl chloride (PVC), etc.); (b) polyamides; (c) fluoropolymers, including but not limited to, polytetrafluoroethylene (PTFE), FEP, ETFE, PFA and MFA; (d) polyolefins (e.g., high density polyethylene, etc.); (e) polyimides; (f) thermoplastic polymers, such as polyaryletherketone (PAEK) and polyether ether ketone (PEEK); (g) polycarbonate; (h) polycarbonate urethane; (i) silicone; (j) acrylic compounds; (k) thermoplastic polyesters; (l) polypropylene; (m) low-density polyethylenes; (n) nylon; (o) sulfone resins; (p) high density polyethylenes; (q) silicone, and silicone-based materials and (r) other synthetic biocompatible polymers; and combinations thereof.

The cannula 102 and tip 120 may comprise the same biocompatible material, or they may be different materials that are members of the same family of polymers. For example, in one embodiment the hollow cannula comprises silicone and the occlusion-resistant tip comprises PEEK. Other components, such as additional tubing, etc., may be used with the occlusion-resistant catheter.

In some embodiments, the hollow cannula 102 and occlusion-resistant tip 120 are molded (e.g., thermobonding) together. In other embodiments, the hollow cannula 102 and occlusion-resistant tip 120 are jointed together by a fastening means, such as a luer-lock, snap, or adhesive, for example. In other embodiments, the hollow cannula 102 and occlusion-resistant tip 120 are bonded together using an adhesive.

In some embodiments, the catheter 100 may have a radiopaque additive. In some embodiments, the catheter 100 may be impregnated or loaded with an additive. In some embodiments, the catheter 100 may be coated with an additive. The occlusion-resistant catheter 100 may be made radiopaque by compounding in radiopaque filler. The additive may be added in using any method known to those skilled in the art. The catheter 100 may be made radiopaque by any other method. The catheter 100 may be composed with additives such as barium, tantalum, or any other radiopaque element. The catheter 100 may be radiopaque in its entirety. The catheter 100 may be radiopaque along a portion of its length. The catheter 100 may be radiopaque for a portion of the circumference, along the its entire length, or a portion of its length. The type of additive and the amount used should not negatively affect the physical and mechanical characteristics of the polymer. Further, the percentage of additive should be sufficient to show up on x-ray and on fluoroscope. For example, thermoplastic polyurethanes can be loaded with up to 40% by weight of radiopaque filler.

The amount and type of radiopaque additive may influence both the effect on physical properties and x-ray response, and thus is dependent on the specific use of the catheter, however, once known can be readily determined by one skilled in the art. For example, barium sulfate has a lower x-ray response than bismuth subcarbonate. It takes more barium sulfate to get the same x-ray response as bismuth subcarbonate. Because the density of barium sulfate is about half that of bismuth subcarbonate, it takes up more volume in the polymer mix. The greater the volume that the radiopaque filler takes up in the polymer mix, the greater the reduction in physical properties. In some embodiments, the occlusion-resistant catheter further includes a radiopaque filler selected from the group consisting of bismuth subcarbonate, barium sulfate, tantalum, and combinations thereof.

In some embodiments, the catheter 100 may have radiopaque markings. The markings may be molded or bonded onto the catheter 100 body. The markings may be attached by any other means. The markings may be any material or combination of materials, including a radiopaque agent. The radiopaque agent may be tantalum or any other radiopaque agent. The markings may indicate the length from the catheter 100 tip. The markings may indicate any other feature or length. The markings may be dots, bands, numbers, or any other shape.

In other embodiments, the material selected for the occlusion-resistant catheter can be coated. In some embodiments, the coatings include a moisture-sensitive polymer that becomes lubricious when wetted by blood. Such coatings may include, but are not limited to, bactericides, antibodies, lubricants, and combinations thereof.

In some examples, the surface of the occlusion-resistant catheter 100 may be treated so that coatings will adhere. In such cases, to achieve good adhesion, the surface of the catheter may be treated. Examples of treatments are chemical etchants, plasma treatments, and corona surface treatments.

The occlusion-resistant catheter may be manufactured by any number of methods, including, but not limited to, injection molding, extrusion, and 3D-printing, for example.

In another aspect, a kit for treating hydrocephalus in a subject is provided. The kit may comprise an occlusion-resistant catheter as described above and instructions for use. In other embodiments, the kit may further include a catheter inserter or stylet. In other embodiments, the kit may further include a right-angle guide. In other embodiments, the catheter may be provided with an attached fitting. In other embodiments, the catheter may be provided with a loose fitting.

One aspect provides a method of treating hydrocephalus in a subject comprising of inserting into the brain of the subject an occlusion-resistant catheter as descried herein such that the hydrocephalus is treated. The term “treatment” or “treating” refers to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

Another aspect provides a method of decreasing pressure in the brain of a subject, the method comprising inserting into the brain of the subject an occlusion-resistant catheter as in any of the preceding claims such that the pressure is decreased in the brain of the subject.

Yet another aspect provides a method of draining cerebrospinal fluid from the brain of a subject comprising inserting into the brain of the subject an occlusion-resistant catheter as in any of the preceding claims such that the cerebrospinal fluid is drained from the brain of the subject.

In some embodiments, the occlusion-resistant catheter is inserted into a ventricle of the brain.

One skilled in the art will readily appreciate that the present application is well adapted to carry out the objects and obtain the ends and advantages mentioned, as well as those inherent therein. The present disclosure described herein is presently representative of preferred embodiments, are exemplary, and are not intended as limitations on the scope of the disclosure. Changes therein and other uses will occur to those skilled in the art which are encompassed within the spirit as defined by the scope of the claims.

While a number of exemplary aspects and embodiments have been discussed above, those of skill in the art will recognize that still further modifications, permutations, additions and sub-combinations thereof of the features of the disclosed embodiments are still possible. It is therefore intended that the following appended claims and claims hereafter introduced are interpreted to include all such modifications, permutations, additions and sub-combinations as are within their true spirit and scope.

What is claimed is:

1. An occlusion-resistant catheter comprising:
a hollow cannula defining a fluid passageway therethrough, the hollow cannula comprising a body portion having a first end and a second end; and
an occlusion-resistant tip positioned at the first end of the body portion, the occlusion-resistant tip comprising at least one port that provides access to an interior lumen, the interior lumen in fluid communication with the fluid passageway of the body portion.

2. The occlusion-resistant catheter according to claim 1 wherein the occlusion-resistant tip further comprises a plurality of protrusions positioned in a helical manner around the tip, the protrusions being positioned around a plurality of ports that allow for fluid to pass into the interior lumen of the tip.

3. The occlusion-resistant catheter according to claim 1 wherein the occlusion-resistant tip further comprises a plurality of protrusions positioned in a spiral manner around the tip, the protrusions being positioned around a plurality of ports that allow for fluid to pass first into the interior lumen of the tip.

4. The occlusion-resistant catheter according to claim 1 wherein the occlusion-resistant tip further comprises a plurality of elongated ports that allow fluid to pass into the interior lumen of the tip.

5. The occlusion-resistant catheter according to claim 4 wherein the plurality of elongated ports are rectangular in shape.

6. The occlusion-resistant catheter according to claim 1 wherein the occlusion-resistant tip further comprises a lattice structure having a plurality of ports that allow fluid to pass into the interior lumen of the tip.

7. The occlusion-resistant catheter according to claim 6 wherein the lattice structure comprises a plurality of layers, each layer including a plurality of ports.

8. The occlusion-resistant catheter according to claim 7 wherein the plurality of ports of each lattice layer are staggered.

9. The occlusion-resistant catheter according to claim 1 wherein the occlusion-resistant tip further comprises a plurality of recesses having a plurality of ports positioned at a bottom of the recesses.

10-11. (canceled)

12. The occlusion-resistant catheter according to claim 1 wherein the occlusion-resistant tip further comprises one or more nested layers, wherein each layer defines an interior lumen and comprises a plurality of recesses, each of the plurality of recesses comprising a plurality of ports positioned at the bottom of the recesses, the ports providing access to the interior lumen of the next nested layer.

13-16. (canceled)

17. The occlusion-resistant catheter according to claim 1 wherein the occlusion-resistant tip comprises a length of about 0.5 cm to about 3.5 cm.

18. (canceled)

19. The occlusion-resistant catheter according to claim 1 in which the catheter is impregnated with antibiotics or antimicrobials.

20. The occlusion-resistant catheter according to claim 1 in which the catheter may be made radiopaque.

21. occlusion-resistant catheter according to claim 1 in which the catheter comprises a biocompatible material.

22. The occlusion-resistant catheter according to claim 21 in which the hollow cannula comprises silicone.

23. The occlusion-resistant catheter according to claim 21 in which the occlusion-resistant tip comprises a thermoplastic polymers.

24. The occlusion-resistant catheter according to claim 1 in which the occlusion-resistant tip is molded to the hollow cannula.

25. The occlusion-resistant catheter according to claim 1 in which the occlusion-resistant tip is bonded to the hollow cannula.

26. (canceled)

27. A method of decreasing pressure in the brain of a subject, the method comprising inserting into the brain of the subject an occlusion-resistant catheter according to claim 1, such that the pressure is decreased in the brain of the subject.

28. A method of draining cerebral spinal fluid from the brain of a subject comprising inserting into the brain of the subject an occlusion-resistant catheter according to claim 1, such that the cerebral spinal fluid is drained from the brain of the subject.

29. (canceled)
